(19) United States
(12) Patent Application Publication
(10) Pub. No.: US 2007/0142475 A1
(43) Pub. Date: Jun. 21, 2007

(54) Delta-amino-gamma-hydroxy-omega-aryl-alkanoic acid amides and use as renin inhibitors

(76) Inventors: Holger Sellner, Therwil (CH); Gerhard Gross, Lorrach (DE); Juergen Klaus Maibaum, Weil-Haltingen (DE); Sylvain Cottens, Witterswil (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/580,295

(22) PCT Filed: Nov. 30, 2004
(86) PCT No.: PCT/EP04/13589

§ 371 Date: May 23, 2006

(30) Foreign Application Priority Data
Dec. 1, 2003 (GB) 0327839.7

(51) Int. Cl. A61K 31/165; C07C 237/42

(52) U.S. Cl. 514/616; 564/152

(57) ABSTRACT

The invention relates to novel δ-amino-γ-hydroxy-ω-aryl-alkanoic acid amides of formula (I) 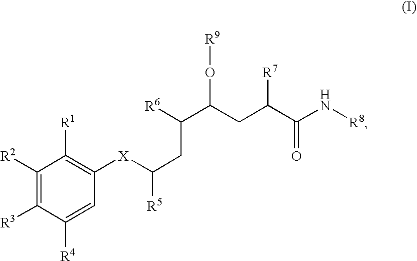 or a pharmaceutically acceptable salt thereof.

The invention relates to novel δ-amino-γ-hydroxy-ω-aryl-alkanoic acid amides of formula (I) 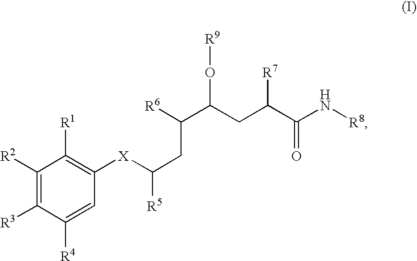
or a pharmaceutically acceptable salt thereof; wherein
R¹, R², R³, R⁴, independently of one another, are hydrogen; halogen; hydroxyl, C₁-C₇-alkanoyloxy, C₁-C₇alkyl; or is
C₁-C₇alkyl that is substituted by: halogen, cyano, hydroxy, C₁-C₇alkanoyl-oxy, C₁-C₇alkoxy,
C₁-C₇alkoxy that is substituted by halogen or by hydroxyl, C₂-C₇alkenyloxy, C₃-C₇-cycloalkoxy, C₁-C₇alkylthio, S-oxidized C₁-C₇alkylthio, amino, N-mono-C₁-C₇alkylamino,
N,N-di-C₁-C₇alkyl-amino, N-C₁-C₇alkanoyl-amino, N—C₁-C₇-alkanesulfonyl-amino, amino that is N,N-disubstituted by C₂-C₇alkylene, by unsubstituted or N′-C₁-C₇alkyl- or N′-C₁-C₇-alkanoyl-aza-C₂-C₇alkylene, by oxa-C₁-C₇alkylene, by thia-C₁-C₇alkylene or by S-oxidized thia-C₁-C₇-alkylene, free or esterified or amidated carboxy, C₃-C₇-cycloalkyl, aryl, heteroaryl, hydrogenated heteroaryl or by oxo; or is
C₁-C₇-alkoxy-C₂-C₇-alkenyl; or C₁-C₇alkoxy; or is
C₁-C₇alkoxy that is substituted by: halogen, cyano, hydroxyl, C₁-C₇alkanoyl-oxy, C₁-C₇-alkoxy, C₁-C₇alkoxy that is substituted by halogen or by hydroxy, C₂-C₇alkenyloxy, C₃-C₇-cycloalkoxy, C₁-C₇alkylthio, S-oxidized C₁-C₇alkylthio, amino, N-mono-C₁-C₇alkylamino,
N,N-di-C₁-C₇alkyl-amino, N—C₁-C₇-alkanoyl-amino, N—C₁-C₇-alkanesulfonyl-amino, amino that is N,N-disubstituted by C₂-C₇-alkylene, by unsubstituted or N′-C₁-C₇alkyl- or N′-C₁-C₇-alkanoyl-aza-C₂-C₇-alkylene, by oxa-C₁-C₇alkylene, by thia-C₁-C₇alkylene or by S-oxidized thia-C₁-C₇alkylene, free or esterified or amidated carboxy, C₃-C₇-cycloalkyl, aryl, heteroaryl, or by hydrogenated heteroaryl; or is
C₂-C₇-alkenyloxy; C₁-C₇alkoxy-C₂-C₇-alkenyloxy; C₃-C₇-cycloalkoxy; C₁-C₇alkanoyl; C₃-C₇-cycloalkyl; aryl; heteroaryl; or hydrogenated heteroaryl; or
R³ together with R₄ form C₂-C₇-alkylenedioxy or a fused-on benzo or cyclohexeno ring;
X is methylene; hydroxymethylene; O; NH; S; SO; or SO₂;
R⁵ is C₁-C₇-alkyl; C₂-C₇-alkenyl; C₃-C₇cycloalkyl; C₃-C₇-cycloalkyl-C₁-C₇alkyl; aryl-C₁-C₇-alkyl; heteroaryl-C₁-C₇alkyl; aryl or heteroaryl;
R⁶ is amino; N-mono-C₁-C₇-amino; N,N-di-C₁-C₇-amino; N—C₁-C₇-alkanoyl-amino; N—C₁-C₇-alkanesulfonyl or represents a group of the formula —NR¹⁰COCHR¹¹NR¹²R¹³, the latter may be present either in the (D)-, (L)- or racemic (D, L)-configuration, but preferably in the L-form;
R⁷ is C₁-C₇alkyl, C₂-C₇-alkenyl; C₃-C₇cycloalkyl; C₃-C₇-cycloalkyl-C₁-C₇alkyl; aryl-C₁-C₇-alkyl; heteroaryl-C₁-C₇alkyl; aryl or heteroaryl;
R⁸ is hydrogen; C₁-C₇alkyl; or is
C₁-C₇alkyl that is substituted by: halogen, cyano, hydroxy, C₁-C₇alkanoyl-oxy, C₁-C₇alkoxy, C₁-C₇-alkoxy that is substituted by halogen or by hydroxyl, C₂-C₇alkenyloxy, C₃-C₇-cycloalkoxy, C₁-C₇alkylthio, S-oxidized C₁-C₇alkylthio, amino, N-mono-C₁-C₇alkylamino, N,N-di-C₁-C₇alkyl-amino, N—C₁-C₇-alkanoyl-amino, N—C₁-C₇-alkanesulfonyl-amino, amino that is N,N-disubstituted by C₂-C₇alkylene, by unsubstituted or N′-C₁-C₇alkyl- or N′-C₁-C₇-alkanoyl-aza-C₂-C₇-alkylene, by oxa-C₁-C₇alkylene, by thia-C₁-C₇alkylene or by S-oxidized thia-C₁-C₇alkylene, free or esterified or amidated carboxy, or is
C₁-C₇alkanoyl; C₃-C₇-cycloalkyl, aryl, heteroaryl, hydrogenated heteroaryl; C₃-C₇-cycloalkyl; aryl; heteroaryl or hydrogenated heteroaryl;
R⁹ represents C₁-C₇alkanoyl, C₁-C₇-alkanesulfonyl or a group of the formula —COCHR¹⁴NR¹¹R¹² which may be present either in the (D)-, (L)- or racemic (D, L)-configuration, but preferably in the L-form; or a group of the formula —CH₂O—COR¹⁵;
R¹⁰ is hydrogen; C₁-C₇alkyl; C₃-C₇-cycloalkyl; C₃-C₇-cycloalkyl-C₁-C₇alkyl; aryl-C₁-C₇-alkyl; heteroaryl-C₁-C₇-alkyl; aryl or heteroaryl;
R¹¹ is hydrogen; C₁-C₇alkyl; aryl-C₁-C₇alkyl; heteroaryl-C₁-C₇alkyl; aryl or heteroaryl;
R¹² and R¹³, independently of another, are hydrogen; C₁-C₇alkyl;
C₁-C₇alkyl that is substituted by: halogen, C₃-C₇-cycloalkyl, aryl, heteroaryl, C₁-C₇-alkoxycarbonyl, C₁-C₇alkylthio, by S-oxidized C₁-C₇-alkylthio, by aminocarbonyl, by N—C₁-C₇-alkanoyl-aminocarbonyl, by N—C₁-C₇alkyl-aminocarbonyl; by N,N-di-C₁-C₇alkyl-aminocarbonyl, or by aminocarbonyl that is disubstituted by C₂-C₇-alkylene; or are C₃-C₇-cycloalkyl; aryl or heteroaryl;
R¹⁴ is hydrogen; C₁-C₇alkyl; aryl-C₁-C₇alkyl; heteroaryl-C₁-C₇alkyl; aryl or heteroaryl; and
R¹⁵ is C₁-C₇-alkyl, aryl-C₁-C₇alkyl; heteroaryl-C₁-C₇alkyl; aryl or heteroaryl.

Salts of compounds having salt-forming groups are especially acid addition salts, salts with bases or, where several salt-forming groups are present, can also be mixed salts or internal salts. Salts are especially the pharmaceutically acceptable or non-toxic salts of compounds of formula I. In a broader sense, the invention relates also to salts which are not suitable for therapeutic purposes and may be used for example in the isolation or purification of free compounds of formula (I) or pharmaceutically acceptable salts thereof. Only salts that are pharmaceutically acceptable and non-toxic are used therapeutically and those salts are therefore preferred.

The compounds of the present invention possess two or more asymmetric centers depending on the choice of the substituents. However, any possible diastereoisomers, enantiomers and geometric isomers, and mixtures thereof, e.g., racemates, are encompassed by the instant invention.

The general terms used hereinbefore and hereinafter have the following meanings, unless defined otherwise.

Halogen is in particular halogen of atomic number not more than 35, such as fluorine, chlorine or bromine, and also includes iodine.

C₁-C₇-Alkanoyl is, for example, formyl or preferably C₂-C₇alkanoyl, such as acetyl, propionyl, butyryl, isobutyryl or pivaloyl. C₂-C₅-alkanoyl is preferred.

C₁-C₇-Alkyl is, in particular, C₁-C₄-alkyl such as methyl, ethyl, n-propyl, isopropyl, n-butyl, isobutyl. Methyl and ethyl are preferred.

C₁-C₇-Alkoxy is, in particular, C₁-C₄-alkoxy such as methoxy, ethoxy, n-propyloxy, isopropyloxy, n-butyloxy and t-butyloxy. Methoxy and ethoxy are preferred.

C₂-C₇-Alkenyl is, in particular C₃-C₇alkenyl and is, for example, 2-propenyl or 1-, 2- or 3-butenyl. C₃-C₅-alkenyl is preferred.

C₃-C₇-Cycloalkoxy is, for example, cyclopropyloxy, cyclobutyloxy, cyclopentyloxy, cyclohexyloxy and cycloheptyloxy. Cyclopropyloxy, cyclopentyloxy and cyclohexyloxy are preferred.

S-Oxidized C₁-C₇-alkylthio is, for example, C₁-C₇-alkanesulfinyl or C₁-C₇alkanesulfonyl.

C₂-C₇-Alkylene is, for example, straight-chain or branched and is in particular methylene, ethylene, propylene and butylene and also 1,2-propylene, 2-methyl-1,3-propylene and 2,2-dimethyl-1,3-propylene. C₂-C₅-alkylene is preferred.

Aza-C₂-C₇-alkylene is, for example, C₂-C₃-alkylene-aza-C₃-C₄-alkylene such as 3-aza-pentylene.

Oxa-C₂-C₇-alkylene is, for example, C₂-C₃-alkylene-oxa-C₃-C₄-alkylene such as 3-oxa-pentylene.

Thia-C₂-C₇-alkylene is, for example, C₂-C₃-alkylene-thia-C₃-C₄-alkylene such as 3-thia-pentylene.

Esterified carboxy is, for example, C₁-C₇alkoxycarbonyl, C₁-C₇alkoxy-C₁-C₇alkoxycarbonyl, C₃-C₇cycloalkyl-C₁-C₇alkoxy-carbonyl, aryl-C₁-C₇-alkoxy-carbonyl or heteroaryl-C₁-C₇ alkoxy-carbonyl.

Amidated carboxy is, for example, aminocarbonyl, N-mono-C₁-C₇alkylaminocarbonyl, N,N-di-C₁-C₇-aminocarbonyl, N—C₁-C₇alkanoyl-aminocarbonyl, N—C₁-C₇alkanesulfonyl-aminocarbonyl, aminocarbonyl that is N,N-disubstituted by C₂-C₇-alkylene, by unsubstituted or N′-C₁-C₇alkyl- or N′-C₁-C₇-alkanoyl-aza-C₂-C₇-alkylene, by oxa-C₁-C₇-alkylene, by thia-C₁-C₇alkylene or by S-oxidized thia-C₁-C₇alkylene.

C₃-C₇-Cycloalkyl is, for example, cyclopropyl, cyclobutyl, cyclopentyl, cyclohexyl and cycloheptyl. Cyclopropyl, cyclopentyl and cyclohexyl are preferred.

Aryl is, for example, phenyl, biphenylyl or naphthyl. Aryl is unsubstituted or substituted, e.g. mono-, di- or tri-substituted, by a substitutent selected from the group consisting of C₁-C₇-alkyl, C₁-C₇alkoxy, hydroxy, cyano, nitro, C₁-C₇-alkanoyloxy, C₁-C₇alkanoyl, halogen and by trifluoromethyl.

Heteroaryl is, for example, optionally benzo-fused 5-membered aza-, diaza-, triaza-, oxadiaza- or tetraaza-aryl radical or a 6-membered aza- or diaza-aryl radical. Appropriate 5-membered heteroaryl radicals are, for example, monoaza-, diaza-, triaza-, tetraaza-, monooxa- or monothia-cyclic aryl radicals, such as pyrrolyl, pyrazolyl, imidazolyl, triazolyl, tetrazolyl, furyl and thienyl, while suitable appropriate 6-membered radicals are in particular pyridyl. Heteroaryl is unsubstituted or substituted, e.g. mono-, di- or tri-substituted, by a substitutent selected from the group consisting of C₁-C₇-alkyl, C₁-C₇-alkoxy, hydroxy, cyano, nitro, C₁-C₇-alkanoyloxy, C₁-C₇alkanoyl, halogen and by trifluoromethyl. Pyrrolyl is, for example, 2- or 3-pyrrolyl. Pyrazolyl is 3- or 4-pyrazolyl. Imidazolyl is 2- or 4-imidazolyl. Triazolyl is, for example, 1,3,5-1H-triazol-2-yl or 1,3,4-triazol-2-yl. Tetrazolyl is, for example, 1,2,3,4-tetrazol-5-yl, furyl is 2- or 3-furyl and thienyl is 2- or 3-thienyl, while suitable pyridyl is 2-, 3- or 4-pyridyl.

Hydrogenated heteroaryl is, for example, optionally benzo-fused 5-membered partially or fully hydrogenated aza-, diaza-, triaza-, oxadiaza- or tetraaza-aryl radical or a 6-membered aza- or diaza-aryl radical. Appropriate hydrogenated 5-membered heteroaryl radicals are, for example, monoaza-, diaza-, triaza-, tetraaza-, monooxa- or monothia-cyclic aryl radicals, such as pyrrolinyl, pyrrolidinyl, pyrazolinyl, pyrazolidinyl, imidazolinyl, imidazoilidinyl, triazolinyl, triazilidinyl, dihydro- or tertahydro-furyl and dihydro- or tertahydro-thienyl, while suitable appropriate 6-membered radicals are in particular pyridinyl or piperidinyl. Hydrogenated heteroaryl is unsubstituted or substituted, e.g. mono-, di- or tri-substituted, by a substitutent selected from the group consisting of C₁-C₇-alkyl, C₁-C₇alkoxy, hydroxy, cyano, nitro, C₁-C₇-alkanoyloxy, C₁-C₇-alkanoyl, halogen and by trifluoromethyl.

C₂-C₇-Alkylenedioxy is, for example, oxy-C₂-C₇alkylenoxy such as oxy-methylene-oxy, oxy-ethylene-oxy, oxy-propylene-oxy or oxy-butylene-oxy. Preferred is C₂-C₅-alkylenedioxy.

The compounds of the present invention have enzyme-inhibiting properties, either by directly blocking the enzyme function, or by releasing an active component of the parent pro-drug molecule which inhibits the function of the target enzyme. In particular, they inhibit, directly and/or indirectly, the action of the natural enzyme renin. The latter passes from the kidneys into the blood where it effects the cleavage of angiotensinogen, releasing the decapeptide angiotensin I which is then cleaved in the lungs, the kidneys and other organs to form the octapeptide angiotensinogen II. The octapeptide increases blood pressure both directly by arterial vasoconstriction and indirectly by liberating from the adrenal glands the sodium-ion-retaining hormone aldosterone, accompanied by an increase in extracellular fluid volume. That increase attributes to the action of angiotensin II. inhibitors of the enzymatic activity of renin bring about a reduction in the formation of angiotensin I. As a result a smaller amount of angiotensin II is produced. The reduced concentration of that active peptide hormone is the direct cause of the hypotensive effect of renin inhibitors.

The action of renin inhibitors is demonstrated inter alia experimentally by means of in vitro tests, i.e. for example by the reduction in the formation of angiotensin I from the natural substrate angiotensinogen, or by the reduction of the cleavage of suitable non-endogenous substrates, being measured in various systems (human plasma, purified human renin together with synthetic or natural renin substrate). Inter alia the following in vitro tests are used:

Inhibition of Human Renin Determined by a Fluorescence Resonance Energy Transfer (FRET) Assay

Recombinant human renin (expressed in Chinese Hamster Ovary cells and purified using standard methods) at 4 nM concentration is incubated with test compound at various concentrations for 1 hour at room temperature in 0.1 M Tris-HCl buffer, pH 7.4, containing 0.05 M NaCl, 0.5 mM EDTA and 0.05% CHAPS. Synthetic peptide substrate Arg-Glu(EDANS)—Ile-His-Pro-Phe-His-Leu-Val-Ile_His_Thr-Lys(DABCYL)-Arg9 is added to a final concentration of 2 μM and increase in fluorescence is recorded at an excitation wave-length of 340 nm and at an emission wave-length of 485 nm in a microplate spectro-fluorimeter. IC50 values are calculated from percentage of inhibition of renin activity as a function of test compound concentration.

Inhibition of Human-Renin Determined by an HPLC Quantification of Enzyme Cleavage Products

Recombinant human renin (expressed in Chinese Hamster Ovary cells and purified using standard methods) at 1 nM concentration is incubated with test compound at various concentrations for 1.5 hours at 37° C. in 0.1 M Tris/HCl pH 7.4 containing 0.05 M NaCl, 0.5 mM EDTA and 0.025% (w/v) CHAPS. Synthetic peptide substrate Ac—Ile-His-Pro-Phe-His-Leu-Val-Ile-His-Asn-Lys-[DY-505-X5] is added to a final concentration of 5 μM. The enzyme reaction is stopped by adding 6 μl of 1.0% TFA. The product of the reaction is separated by HPLC and quantified by spectrophotometric measurement at 505 nM wave-length. IC50 values are calculated from percentage of inhibition of renin activity as a function of test compound concentration.

Inhibition of Human Renin by a Scintillation Proximity Assay (SPA)

Recombinant human renin (expressed in Chinese Hamster Ovary cells and purified using standard methods) at 3.3 nM concentration, 1251-NVP-AJ1891-NX-1 (0.27 □Ci/ml) and streptavidin-SPA (0.67 mg/ml) beads are incubated with test compound at various concentrations for 2.0 hours at room temperature in 0.1 M Tris/HCl pH 7.4 containing 0.5 M NaCl and 0.5% (w/v) Brij35. At the end of the incubation time, the plates are centrifuged (55 g, 60 seconds) and counted in a Wallac MicroBeta reader. IC50 values are calculated from percentage of displacement of radioligand binding to renin as a function of test compound concentration.

In Vivo Test Systems:

In animals deficient in salt, renin inhibitors bring about a reduction in blood pressure. Human renin differs from the renin of other species. In order to test inhibitors of human renin, primates (marmosets, Callithrix jacchus) are used, because human renin and primate renin are substantially homologous in the enzymafically active region. Inter alia the following in vivo test is used: the test compounds are tested on normotensive marmosets of both sexes having a body weight of approximately 350 g that are conscious, allowed to move freely and in their normal cages. The blood pressure and heart rate are measured via a catheter in the descending aorta and recorded radiometrically. The endogenous release of renin is stimulated by the combination of a 1-week low-salt diet and a single intramuscular injection of furosemide (5-(aminosulfonyl)-4-chloro-2-[(2-furanylmethyl)amino]benzoic acid) (5 mg/kg). 16 hours after the injection of furosemide the test compounds are administered either directly into the femoral artery using an injection cannula or, in the form of a suspension or solution, via an oesophageal tube into the stomach, and their action on the blood pressure and heart rate are evaluated. In the in vivo test described, the compounds of the present invention have hypotensive action at doses of from approximately 0.003 to approximately 0.3 mg/kg i.v. and at doses of from approximately 0.31 to approximately 30 mg/kg p.o.

The compounds of the present invention also have the property of regulating, especially reducing, intra-ocular pressure.

The extent of the reduction in intra-ocular pressure after administration of a pharmaceutical active ingredient of formula (I) according to the present invention can be determined, for example, in animals, for example rabbits or monkeys. Two typical experimental procedures that illustrate the present invention, but are not intended to limit it in any way, are described hereinafter.

The in vivo test on a rabbit of the “Fauve de Bourgogne” type to determine the intra-ocular-pressure-reducing activity of topically applied compositions can be designed, for example, as follows. The intra-ocular pressure (IOP) is measured using an aplanation tonometer both before the experiment and at regular intervals of time. After a local anaesthetic has been administered, the suitably formulated test compound is applied topically in a precisely defined concentration (e.g. 0.000001-5% by weight) to one eye of the animal in question. The contralateral eye is treated, for example, with physiological saline. The measured values thus obtained are evaluated statistically.

The in vivo tests on monkeys of the species Macaca Fascicularis to determine the intra-ocular-pressure-reducing activity of topically applied compositions can be carried out, for example, as follows. The suitably formulated test compound is applied in a precisely defined concentration (e.g. 0.000001-5% by weight) to one eye of each monkey. The other eye of the monkey is treated correspondingly, for example with physiological saline. Before the start of the test the animals are anaesthetised with intramuscular injections of, for example, ketamine. At regular intervals of time, the intra-ocular pressure (IOP) is measured. The test is carried out and evaluated in accordance with the rules of “good laboratory practice” (GLP).

Accordingly, the compounds of the present invention can be used in the prevention of, treatment of or delay of progression to overt hypertension, atherosclerosis, unstable coronary syndrome, congestive heart failure, cardiac hypertrophy, cardiac fibrosis, cardiomyopathy, postinfarction, (acute and chronic) renal failure, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, alzheimers, dementia, anxiety states and cognitive disorders.

Accordingly, the compounds of the present invention can be used in the prevention of, treatment of or delay of progression to overt hypertension, congestive heart failure, cardiac hypertrophy, cardiac fibrosis, cardiomyopathy, postinfarction, (acute and chronic) renal failure, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, anxiety states and cognitive disorders.

The groups of compounds mentioned below are not to be regarded as exclusive; rather, for example in order to replace general definitions with more specific definitions, parts of those groups of compounds can be interchanged or exchanged for the definitions given above, or omitted, as appropriate.

Preferred R¹ is hydrogen, C₁-C₇alkyl or C₁-C₇alkoxy.

Preferred R² and R³ are C₁-C₇-alkoxy or C₁-C₇alkoxy-C₁-C₇-alkoxy.

Preferred R⁴ is hydrogen, C₁-C₇-alkyl or C₁-C₇alkoxy.

Preferred R⁵ and R⁷ are C₁-C₇-alkyl.

Preferred R⁵ is amino.

Preferred R⁸ is amino-carbonyl-C₁-C₇-alkyl.

Preferred R⁹ is C₁-C₇alkanoyl, a group of the formula —COCHR¹⁴NR¹¹R¹² which may be present either in the (D)-, (L)- or racemic (D, L)-configuration, but preferably in the L-form; or a group of the formula —CH₂O—COR¹⁵; and R¹⁴ is hydrogen, C₁-C₇-alkyl or phenyl-C₁-C₄-alkyl; R¹¹ and R¹², independently of one another, are hydrogen, C₁-C₇-alkyl or phenyl-C₁-C₄-alkyl; and R¹⁵ is C₁-C₇-alkyl or phenyl-C₁-C₄-alkyl.

Preferred R¹⁰, R¹¹, R¹², R¹³ and R¹⁴ are hydrogen, C₁-C₇alkyl or phenyl-C₁-C₄-alkyl.

Preferred R¹⁵ is C₁-C₇alkyl or phenyl-C₁-C₄-alkyl.

Preferred X is methylene.

The invention relates especially to a compound of formula (I), or a pharmaceutically acceptable salt thereof; wherein

R¹ is hydrogen, C₁-C₇-alkyl or C₁-C₇alkoxy; R² is C₁-C₇-alkoxy or C₁-C₇alkoxy-C₁-C₇alkoxy;

R³ is C₁-C₇alkoxy or C₁-C₇-alkoxy-C₁-C₇-alkoxy; R⁴ is hydrogen, C₁-C₇alkyl or C₁-C₇-alkoxy;

R⁵ is C₁-C₇alkyl; R⁶ is amino; R⁷ is C₁-C₇-alkyl; R⁸ is amino-carbonyl-C₁-C₇-alkyl; R⁹ is C₁-C₇-alkanoyl, a group of the formula —COCHR¹⁴NR¹¹R¹² which may be present either in the (D)-, (L)- or racemic (D, L)-configuration, but preferably in the L-form; or a group of the formula —CH₂O—COR¹⁵; and R¹⁴ is hydrogen, C₁-C₇-alkyl or phenyl-C₁-C₄-alkyl; R¹² and R¹³,

independently of one another, are hydrogen, C₁-C₇alkyl or phenyl-C₁-C₄-alkyl; and R¹⁵ is C₁-C₇alkyl or phenyl-C₁-C₄alkyl; and X is methylene.

The invention relates especially to a compound of formula (I A) 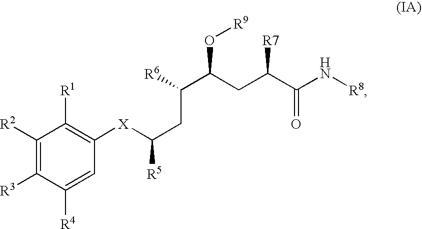
wherein the variables R¹ to R¹⁵ and X have all meanings as given above; or a pharmaceutically acceptable salt thereof.

The invention relates especially to a compound of formula (I A) or a pharmaceutically acceptable salt thereof, wherein

R¹ and R⁴ are hydrogen; R² is C₁-C₄-alkoxyl-C₁-C₄-alkoxy, such as 3-methoxy-propyloxy; R³ is C₁-C₄-alkoxy, such as methoxy; R⁵ and R⁷, independently of one another, are C₁-C₇alkyl, such as isopropyl; R⁶ is amino; R⁸ is aminocarbonyl-C₁-C₄-alkyl, such as 2-amino-2,2-dimethylethyl; R⁹ is C₁-C₄-alkanoyl or a group of the formula —COCHR¹⁴NR¹²R¹³ wherein R¹⁴ is C₁-C₄-alkyl, such as isopropyl or isobutyl, or phenyl-C₁-C₂-alkyl, such as benzyl, R¹² and R¹³ are hydrogen and X is methylene.

The invention relates especially to a compound of formula (I B) or a pharmaceutically acceptable salt thereof, wherein 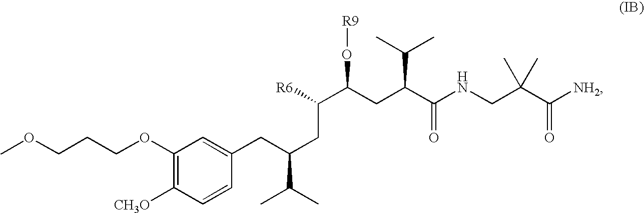
or a pharmaceutically acceptable salt thereof, wherein R⁶ and R⁹ have the meanings as given above in each case.
The invention relates especially to a compound of formula (I C) or a pharmaceutically acceptable salt thereof, wherein 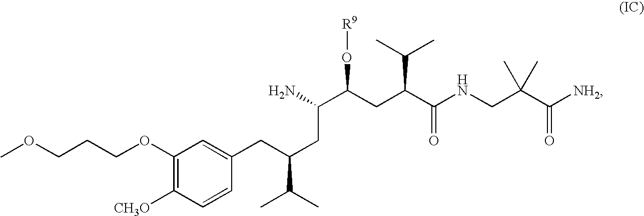
or a pharmaceutically acceptable salt thereof, wherein R⁹ is C₁-C₄-alkanoyl or a group of the formula —COCHR¹⁴NH₂ wherein R¹⁴ is C₁-C₄-alkyl, such as isopropyl or isobutyl, or phenyl-C₁-C₂-alkyl, such as benzyl.

When hereinbefore or hereinafter reference is made to a compound of formula (I), a compound of formula (I A), (I B) and (I C) is likewise encompassed.

The invention relates specifically to the compounds of formula I mentioned in the Examples and to the salts thereof, especially the pharmaceutically acceptable salts thereof.

The present invention also relates to the manufacture of a compound of formula (I) or (I A) or (I B) or (I C), respectively, or a salt thereof, wherein the variables R¹ to R⁵ and R⁷ to R¹⁵ and X have the meanings as defined above and R⁶ is amino, comprising
reducing a compound of formula (II a) 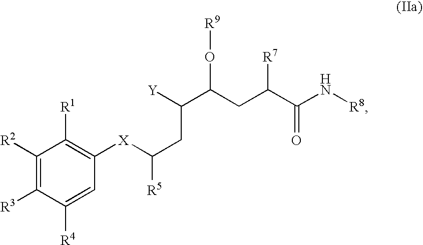
wherein Y is azido (N₃), or a salt thereof and isolating a compound of formula (I) or a salt thereof.

The reduction is carried out in the presence of a hydrogenation catalyst.

The present invention also relates to the manufacture of a compound of formula (I) or (I A) or (I B) or (I C), respectively, or a salt thereof, wherein the variables R¹ to R⁸ and R¹⁰ to R¹⁵ and X have the meanings as defined above, comprising
reacting a compound of formula (II b) 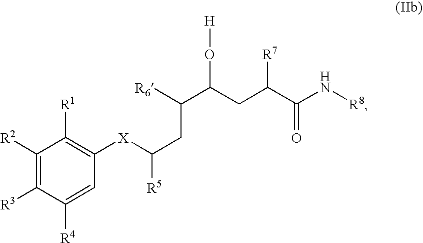
wherein R^{6′} is protected amino or N-mono-C₁-C₇-amino; N,N-di-C₁-C₇-amino; N—C₁-C₇-alkanoyl-amino; or represents a group of the formula —NR¹⁰COCHR¹¹NR²R³, the latter may be present either in the (D)-, (L)- or racemic (D, L)-configuration, but preferably in the L-form; R¹² is hydrogen and R¹³ is an amino protecting, or a salt thereof
with a compound of formula (II c) R⁹—Y¹, wherein Y¹ is hydroxy or a reactive group; or a salt thereof;
removing the corresponding protecting group(s); and
isolating a compound of formula (I) or a salt thereof.

The reactions described herein above and herein below are carried out in a manner known per se, for example in the absence or, usually, in the presence of a suitable solvent or diluent or a mixture thereof, the operation being carried out as necessary with cooling, at room temperature or with heating, for example in a temperature range of approximately from −80° C. to the boiling temperature of the reaction medium, especially from about −10° to about +200° C., and, if necessary, in a closed vessel, under pressure, in an inert gas atmosphere and/or under anhydrous conditions.

The reduction of a compound of formula (II a) is carried out, for example, by hydrogenation in the presence of a hydrogenation catalyst.

A catalyst suitable for hydrogenation are metals, for example nickel, iron, cobalt or ruthenium, or noble metals or their oxides, such as palladium or rhodium or their oxides, optionally supported on a suitable carrier, such as barium sulfate, aluminium oxide or active carbon, or in the form of skeleton catalysts, for example Raney nickel, but especially homogeneous or heterogeneous metal- or noble metal-ligand complexes. A preferred catalyst is Pd/C.

Such catalysts are especially complexes of ruthenium or ruthenium salts, such as Ru(II) halides, such as RuCl₂, Ru₂Cl₂ or RuHCl, optionally halogenated Ru(II) lower alkanoylates, such as Ru(OAc)₂ or Ru(OOC—CF₃)₂, with (S)-bis(2,2′-diphenylphosphino)-1,1′-binaphthyl (S—BINAP) or derivatives thereof which contain instead of phenyl substituted phenyl radicals, such as p-tolyl or p-methoxyphenyl, and also ruthenium complexes with (S)-bis(2,2′-diphenyl-phosphino)-5,5′-dimethyl-diphenyl and the like. Hydrogenation with complexes of that type is preferably carried out inert solvents or mixtures of solvents e.g. alcohols, such as lower alkanols, or alkyl halides, such as methylene chloride, in a pressure range of approximately from 1 to 100 bar, preferably from 20 to 30 bar, and in a temperature range of approximately from 100 to 80° C., preferably from 15° to 25° C. The hydrogenation is carried out at temperatures of from 0° C. to 250° C., preferably from room temperature to about 100° C. and at hydrogen pressures of from 1 to 200 bar.

The reaction of a compound of formula (II b) with a compound of formula (II c) is carried out, for example, following methods known per se.

For the manufacture of a compound of formula (I), wherein R⁹ is C₁-C₇alkanoyl, C₁-C₇ alkanesulfonyl or a group of the formula —COCHR¹⁴NR¹¹R¹² which may be present either in the (D>, (L) or racemic (D, L)-configuration, but preferably in the L-form; the reaction of a compound of formula (II b) with a compound of formula (II c) is an acylation, while, for manufacture of a compound of formula (I), wherein R⁹ is a group of the formula —CH₂O—COR¹⁵; the reaction of a compound of formula (II b) with a compound of formula (II c) is an etherification.

A corresponding acylation is carried out, for example, in the presence of a suitable base. Suitable bases are, for example, alkali metal hydroxides, hydrides, amides, alkanolates, carbonates, triphenylmethylides, di-lower alkylamides, aminoalkylamides or lower alkylsilylamides, naphthaleneamines, lower alkylamines, basic heterocycles, ammonium hydroxides, and carbocyclic amines. Examples which may be mentioned are sodium hydroxide, sodium hydride, sodium amide, sodium methoxide, sodium ethoxide, potassium tert-butoxide, potassium carbonate, lithium triphenylmethylide, lithium diisopropylamide, potassium 3-(aminopropyl)amide, potassium bis(trimethylsilyl)amide, dimethylaminonaphthalene, di- or triethylamine, or ethyldiisopropylamine, N-methylpiperidine, pyridine, benzyltrimethylammonium hydroxide, 1,5-diazabicyclo[4.3.0]non-5-ene (DBN) and 1,8-diaza-bicyclo[5.4.0]undec-7-ene (DBU).

A reactive group Y¹ is, for example, halogen such as chloro, bromo or iodo, substituted sulfonyl, or a group of formula R⁹—CO—O— or R⁹—SO₂—O—. The corresponding aclyation is carried out for example in the presence of one of the customary condensing agents. Customary condensing agents are, for example, carbodiimides, for example diethyl-, dipropyl- or N-ethyl-N′-(3-dimethylaminopropyl)carbodiimide or in particular dicyclohexylcarbodiimide, and also suitable carbonyl compounds, for example carbonyldiimidazole, 1,2-oxazolium compounds, for example 2-ethyl-5-phenyl-1,2-oxazolium-3′-sulfonate and 2-tert-butyl-5-methylisoxazolium perchlorate, or a suitable acylamino compound, for example 2-ethoxy-1-ethoxycarbonyl-1,2-dihydroquinoline, and also activated phosphoric acid derivatives, for example diphenylphosphoryl azide, diethylphosphoryl cyanide, phenyl N-phenylphosphoramidochloridates, bis(2-oxo-3-oxazolidinyl)phosphinoyl chloride or 1-benzotriazolyloxy-tris(dimethylamino)phosphonium hexafluorophosphate.

If desired, an organic base is added, for example a tri-lower alkylamine having bulky radicals, for example ethyldiisopropylamine, or a heterocylic base, for example pyridine, 4-dimethylaminopyridine or preferably N-methylmorpholine.

A corresponding etherification is carried out, for example, following methods known per se. The etherification can be carried out, for example, in the presence of a base, an alkali metal hydride, hydroxide or carbonate, or of an amine. Conversely, corresponding ethers, such as lower alkoxy compounds, can be cleaved, for example, by means of strong acids, such as mineral acids, for example the hydrohalic acids hydrobromic or hydriodic acid, which may advantageously be present in the form of pyridinium halides, or by means of Lewis acids, for example halides of elements of main group III or the corresponding sub-groups. These reactions can be carried out, if necessary, with cooling or warming, for example in a temperature range from about −2001 to about 100° C., in the presence or absence of a solvent or diluent, under inert gas and/or under pressure and, if appropriate, in a closed vessel.

The removal of amino protection groups is carried out following for example methods known per se, especially by using methods that are applied in the manufacture of peptides or proteins. For example, the Boc group is removed in the presence of an acid, such as hydrochloric acid, in an inert solvent or a mixture of solvents, such as in an ether, for example dioxane.

The isolation of a compound of formula (I) is carried out according to conventional isolation methods, such as by crystallizing the resulting compound of formula (I) from the reaction mixture or by chromatography of the reaction mixture.

The process for the manufacture of compounds of formula (I) and salts thereof can be, for example, illustrated by the working examples which likewise together with the method of preparing the compounds of formula (I) are another aspect of the present invention.

The starting material of compounds of formulae (II a), (II b) and (II c) are known or can be prepared by using and applying methods known per se in the art. The manufacture of compounds of formula (II a) is, for example, described in the working examples. Compounds of formula (II a) and (II b) can be prepared, for example, by using the methods as described in EP 678503A1; the corresponding subject matter relating to the manufacture of said starting material is herein incorporated by reference.

In view of the close relationship between the novel compound in the free form and in the form of its salts, in the preceding text and below the free compound or its salts may correspondingly and advantageously also be understood as meaning the corresponding salts or the free compound.

Salts of compounds of formula (I) can be prepared in a manner known per se. For example, acid addition salts of compounds of formula (I) are obtained by treatment with an acid or a suitable ion exchange reagent. Acid addition salts can be converted into the free compounds in customary manner, e.g. by treatment with a suitable basic agent.

Resulting acid addition salts can be converted into other salts in a manner known per se, for example by treatment with a suitable metal salt, such as a sodium, barium or silver salt, of a different acid in a suitable solvent in which an inorganic salt formed is insoluble and is therefore eliminated from the reaction equilibrium.

The compounds of formula (I), including a salt thereof, may also be obtained in the form of a hydrate or may include the solvent used for crystallisation (solvates).

As a result of the close relationship between the novel compounds in free form and in the form of their salts, hereinabove and hereinbelow any reference to the free compounds and their salts is to be understood as including also the corresponding salts and free compounds, respectively, as appropriate and expedient.

The invention especially relates to a combination, such as a combined preparation or pharmaceutical composition, respectively, comprising a compound of formula (I) or a pharmaceutically acceptable salt thereof and at least one therapeutic agent selected from the group consisting of

(i) an AT₁-receptor antagonist or a pharmaceutically acceptable salt thereof,

(ii) an angiotensin converting enzyme (ACE) inhibitor or a pharmaceutically acceptable salt thereof,

(iii) a beta blocker or a pharmaceutically acceptable salt thereof,

(iv) a calcium channel blocker or a pharmaceutically acceptable salt thereof,

(v) an aldosterone synthase inhibitor or a pharmaceutically acceptable salt thereof,

(vi) an aldosterone receptor antagonist or a pharmaceutically acceptable salt thereof,

(vii) a dual angiotensin converting enzyme/neutral endopetidase (ACE/NEP) inhibitor or a pharmaceutically acceptable salt thereof,

(viii) an endothelin receptor antagonist or a pharmaceutically acceptable salt thereof,

(ix) a diuretic or a pharmaceutically acceptable salt thereof;

(x) a neutral endopeptidase (NEP) inhibitor or a pharmaceutically acceptable salt thereof;

(xi) an inhibitors of the Na—K-ATPase membrane pump or a pharmaceutically acceptable salt thereof;

(xii) an antidiabetic agent or a pharmaceutically acceptable salt thereof;

(xiii) a hypolipidemic agent or a pharmaceutically acceptable salt thereof; and

(xiv) an anti-obesity agent or a pharmaceutically acceptable salt thereof.

The invention especially relates to a combination, such as a combined preparation or pharmaceutical composition, respectively, comprising a compound of formula (I) or a pharmaceutically acceptable salt thereof and at least one therapeutic agent selected from the group consisting of

(i) an AT₁-receptor antagonist or a pharmaceutically acceptable salt thereof,

(ii) an angiotensin converting enzyme (ACE) inhibitor or a pharmaceutically acceptable salt thereof,

(iii) a beta blocker or a pharmaceutically acceptable salt thereof,

(iv) a calcium channel blocker or a pharmaceutically acceptable salt thereof,

(v) an aldosterone synthase inhibitor or a pharmaceutically acceptable salt thereof,

(vi) an aldosterone receptor antagonist or a pharmaceutically acceptable salt thereof,

(vii) a dual angiotensin converting enzyme/neutral endopetidase (ACE/NEP) inhibitor or a pharmaceutically acceptable salt thereof,

(viii) an endothelin receptor antagonist or a pharmaceutically acceptable salt thereof,

(ix) a diuretic or a pharmaceutically acceptable salt thereof.

The combination according to the present invention likewise comprises at least one pharmaceutically acceptable carrier.

The term “at least one therapeutic agent” shall mean that in addition to the compound of formula (I) one or more, for example two, furthermore three, active ingredients as specified according to the present invention can be combined.

AT₁-receptor antagonists (also called angiotensin II receptor antagonists) are understood to be those active ingredients that bind to the AT₁-receptor subtype of angiotensin II receptor but do not result in activation of the receptor. As a consequence of the inhibition of the AT₁ receptor, these antagonists can, for example, be employed as antihypertensives or for treating congestive heart failure.

The class of AT₁ receptor antagonists comprises compounds having differing structural features, essentially preferred are the non-peptidic ones. For example, mention may be made of the compounds that are selected from the group consisting of valsartan (cf. EP 443983), losartan (cf. EP253310), candesartan (cf. 459136), eprosartan (cf. EP 403159), irbesartan (cf. EP454511), olmesartan (cf. EP 503785), tasosartan (cf. EP539086), telmisartan (cf. EP 522314), the compound with the designation E-1477 of the following formula 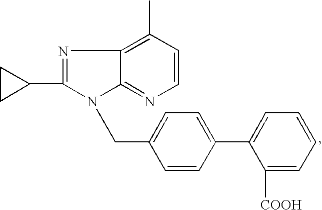
the compound with the designation SC-52458 of the following formula 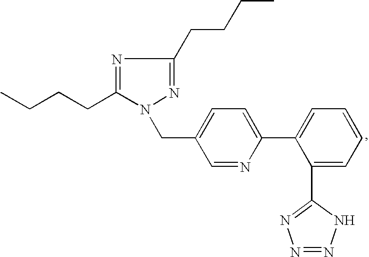
and the compound with the designation the compound ZD-8731 of the following formula 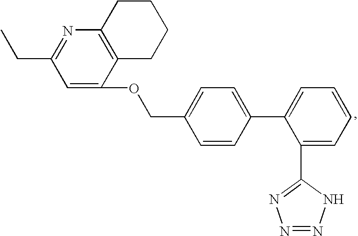
or, in each case, a pharmaceutically acceptable salt thereof.

Preferred AT₁-receptor antagonist are those agents that have been marketed, most preferred is valsartan or a pharmaceutically acceptable salt thereof.

The interruption of the enzymatic degradation of angiotensin I to angiotensin II with so-called ACE-inhibitors (also called angiotensin converting enzyme inhibitors) is a successful variant for the regulation of blood pressure and also a therapeutic method for the treatment of congestive heart failure.

The class of ACE inhibitors comprises compounds having differing structural features. For example, mention may be made of the compounds which are selected from the group consisting alacepril, benazepril, benazeprilat, captopril, ceronapril, cilazapril, delapril, enalapril, enalaprilat, fosinopril, imidapril, lisinopril, moexipril, moveltopril, perindopril, quinapril, quinaprilat, ramipril, ramiprilat, spirapril, temocapril, trandolapril and zofenopril, or, in each case, a pharmaceutically acceptable salt thereof.

Preferred ACE inhibitors are those agents that have been marketed, most preferred are benazepril, enalapril, lisinopril and especially ramipril.

A beta blocker in said combination preferably is a representative selected from the group consisting of a selective β1-blocker, such as atenolol, bisoprolol (especially the fumarate thereof, metoprolol (especially the hemi-(R,R)fumarate or fumarate thereof), furthermore, acetutolol (especially the hydrochloride thereof), esmolol (especially the hydrochloride thereof), celiproplol (especially the hydrochloride thereof), taliprolol, or acebutolol (especially the hydrochloride thereof), a non-selective β-blocker, such as oxprenolol (especially the hydrochloride thereof), pindolol, furthermore, propanolol (especially the hydrochloride thereof, bupranolol (especially the hydrochloride thereof), penbutolol (especially the sulphate thereof), mepindolol (especially the sulphate thereof), carteolol (especially the hydrochloride thereof) or nadolol, and a β-blocker with α-blocking activity such as carvedilol; or in each case, a pharmaceutically acceptable salt thereof.

The class of calcium channel blockers (CCBs) essentially comprises dihydropyridines (DHPs) and non-DHPs such as diltiazem-type and verapamil-type CCBs. A CCB useful in said combination is preferably a DHP representative selected from the group consisting of amlodipine, felodipine, ryosidine, isradipine, lacidipine, nicardipine, nifedipine, niguldipine, niludipine, nimodipine, nisoldipine, nitrendipine, and nivaldipine, and is preferably a non-DHP representative selected from the group consisting of flunarizine, prenylamine, diltiazem, fendiline, gallopamil, mibefradil, anipamil, tiapamil and verapamil, and in each case, a pharmaceutically acceptable salt thereof. All these CCBs are therapeutically used, e.g. as anti-hypertensive, anti-angina pectoris or anti-arrhythmic drugs. Preferred CCBs comprise amlodipine, diltiazem, isradipine, nicardipine, nifedipine, nimodipine, nisoldipine, nitrendipine, and verapamil, or, e.g. dependent on the specific CCB, a pharmaceutically acceptable salt thereof. Especially preferred as DHP is amlodipine or a pharmaceutically acceptable salt, especially the besylate thereof, furthermore the maleate, thereof. An especially preferred representative of non-DHPs is verapamil or a pharmaceutically acceptable salt, especially the hydrochloride, thereof.

Aldosterone synthase is an enzyme that converts corticosterone to aldosterone by hydroxylating corticosterone to form 18-OH-corticosterone and 18-OH-corticosterone to aldosterone. The class of aldosterone synthase inhibitors is known to be applied for the treatment of hypertension and primary aldosteronism comprises both steroidal and non-steroidal aldosterone synthase inhibitors, the later being most preferred.

Preference is given to commercially available aldosterone synthase inhibitors or those aldosterone synthase inhibitors that have been approved by the health authorities.

The class of aldosterone synthase inhibitors comprises compounds having differing structural features. For example, mention may be made of the compounds which are selected from the group consisting of anastrozole, fadrozole (including the (+)-enantiomer thereof), as well as exemestane, or, in each case where applicable, a pharmaceutically acceptable salt thereof.

The most preferred non-steroidal aldosterone synthase inhibitor is the (+)enantiomer of fadrozole (U.S. Pat. Nos. 4,617,307 and 4,889,861) of formula 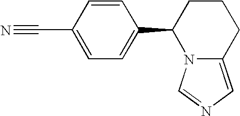
or a pharmaceutically acceptable salt thereof, for example, the hydrochloride thereof.

A preferred steroidal aldosterone receptor antagonist is eplerenone (cf. EP 122232 A) of the formula 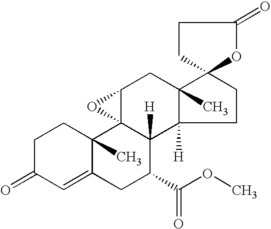
or spironolactone.

Compounds having an inhibitory effects on both angiotensin converting enzyme and neutral endopetidase, so-called dual ACE/NEP inhibitors, can be used for the treatment of cardiovascular pathologies.

A preferred dual angiotensin converting enzyme/neutral endopetidase (ACE/NEP) inhibitor is, for example, omapatrilat (cf. EP 629627), fasidotril or fasidotrilat (cf. EP 419327), or Z 13752A (cf. WO 97/24342) or, if appropriate, a pharmaceutically acceptable salt thereof. A preferred endothelin antagonist is, for example, bosentan (cf. EP 526708 A), enrasentan (cf. WO 94/25013), atrasentan (cf. WO 96/06095), especially atrasentan hydrochloride, darusentan (cf. EP 785926 A), BMS 193884 (cf. EP 702012 A), sitaxentan (cf. U.S. Pat. No. 5,594,021), especially sitaxsentan sodium, YM 598 (cf. EP 882719 A), S 0139 (cf. WO 97/27314), J 104132 (cf. EP 714897 A or WO 97/37665), furthermore, tezosentan (cf. WO 96/19459), or in each case, a pharmaceutically acceptable salt thereof.

A diuretic is, for example, a thiazide derivative selected from the group consisting of amiloride, chlorothiazide, hydrochlorothiazide, methylchlorothiazide, and chlorothalidon. The most preferred is hydrochlorothiazide.

An inhibitors of the Na—K-ATPase membrane pump is, for example, digoxin.

An antidiabetic agent or a pharmaceutically acceptable salt thereof, is, for example, insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; peroxisome proliferator-activated receptor (PPAR) ligands; protein tyrosine phosphatase-1B (PTP-1B) inhibitors such as PTP-112; GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose cotransporter inhibitors such as T-1095; glycogen phosphorylase A inhibitors such as BAY R3401; biguanides such as mefformin; alpha-glucosidase inhibitors such as acarbose; GLP-1 (glucagon like peptide-1), GLP-1 analogs such as Exendin-4 and GLP-1 mimetics; and DPPIV (dipeptidyl peptidase IV) inhibitors such as LAF237.

Other specific anti-diabetic compounds are described by Patel Mona in Expert Opin Investing Drugs, 2003, 12(4), 623-633, in the FIGS. 1to 7+L, which are herein incorporated by reference.

A hypolipidemic agent or a pharmaceutically acceptable salt thereof is, for example, 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin.

An anti-obesity agent is, for example, orlistat.

A compound of the present invention may be administered either simultaneously, before or after the other active ingredient, either separately by the same or different route of administration or together in the same pharmaceutical formulation.

In accordance with the foregoing the present invention also provides a therapeutic combination, e.g., a kit, kit of parts, e.g., for use in any method as defined herein, comprising a compound of formula (I), or a pharmaceutically acceptable salt thereof, to be used concomitantly or in sequence with at least one pharmaceutical composition comprising at least one other therapeutic agent, preferably selected from anti-diabetic agents, hypolipidemic agents, anti-obesity agents or anti-hypertensive agents. The kit may comprise instructions for its administration.

Similarly, the present invention provides a kit of parts comprising: (i) a pharmaceutical composition of the invention; and (ii) a pharmaceutical composition comprising at least one therapeutic agent, especially a compound selected from the above specified combination partners, such as an anti-diabetic, a hypolipidemic agent, an anti-obesity agent, an anti-hypertensive agent, or a pharmaceutically acceptable salt thereof, in the form of two separate units of the components (i) to (ii).

Likewise, the present invention provides a method as defined above comprising co-administration, e.g., concomitantly or in sequence, of a therapeutically effective amount of a compound of formula (I), or a pharmaceutically acceptable salt thereof, and at least one therapeutic agent, especially said drug substance being selected from the above specified combination partners, such as an anti-diabetic, a hypolipidemic agent, an anti-obesity agent or an anti-hypertensive agent, e.g., as indicated above.

Preferably, a compound of the invention is administered to a mammal in need thereof.

Preferably, a compound of the invention is used for the treatment of a disease which responds to modulation of renin activity.

Thus, the present invention also relates to a compound of formula (I) for use as a medicament,

The present invention likewise relates to method for the inhibition of renin activity in mammals or for the prevention of, treatment of or delay of progression to overt to a disease or condition as specified above, which method comprises administering to a mammal in need thereof a therapeutically effective amount of a compound of a compound of formula (I) or a pharmaceutically acceptable salt thereof, either alone or together with at least therapeutic agent.

The structure of the active agents identified by generic or trade names may be taken from the actual edition of the standard compendium “The Merck Index” or from databases, e.g. LifeCycle Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

The new compounds of formula (I) may be present for example in the form of pharmaceutical preparations which comprise a therapeutically effective amount of active substance, if necessary together with inorganic or organic, solid or liquid, pharmaceutically acceptable carriers, and which are suitable for enteral, for example oral or parenteral, administration, especially for the prevention and treatment of a condition or disease as described hereinbefore and hereinafter. The present pharmaceutical preparations which, if so desired, may contain further pharmacologically active substances are prepared in a manner known per se, for example by means of conventional mixing, granulating, coating, dissolving or lyophilizing processes, and contain from about 0.1% to 100%, especially from about 1% to about 50%, of the lyophilisates to about 100% of the active substance.

The invention relates likewise to the use of compounds of formula (I), preferably for the preparation of pharmaceutical compositions, especially for the prevention and treatment of a condition or disease as described hereinbefore and hereinafter.

The invention relates likewise to method for the prevention or treatment of a condition or disease as disclosed hereinbefore and hereinafter comprising administering to a patient (including human) in need thereof an effective amount of a compound of formula (I) or a pharmaceutically acceptable salt thereof.

The present invention further provides pharmaceutical compositions comprising a therapeutically effective amount of a pharmacologically active compound of the instant invention, alone or in combination with one or more pharmaceutically acceptable carriers.

The dosage may depend on various factors, such as the route of administration, species, age and/or condition of the individual. The daily doses to be administered lie between about 0.25 and about 10 mg/kg in the case of oral administration and preferably between about 20 mg and about 500 mg for warm-blooded animals with a body weight of about 70 kg.

The following examples illustrate the invention described above; however, they are not intended to limit its extent in any manner. Temperatures are indicated in degrees Celsius.

The process for the manufacture of compounds of formula (I) and salts thereof can be, for example, illustrated by means of the following reaction scheme:

Hydrogenation of azide group to free primary amine

### EXAMPLE 1

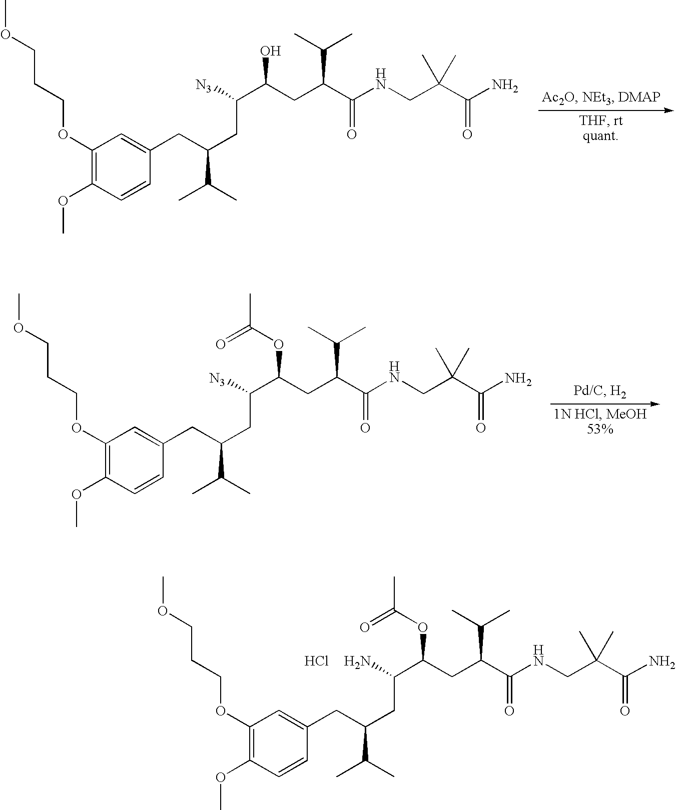

### EXAMPLE 2

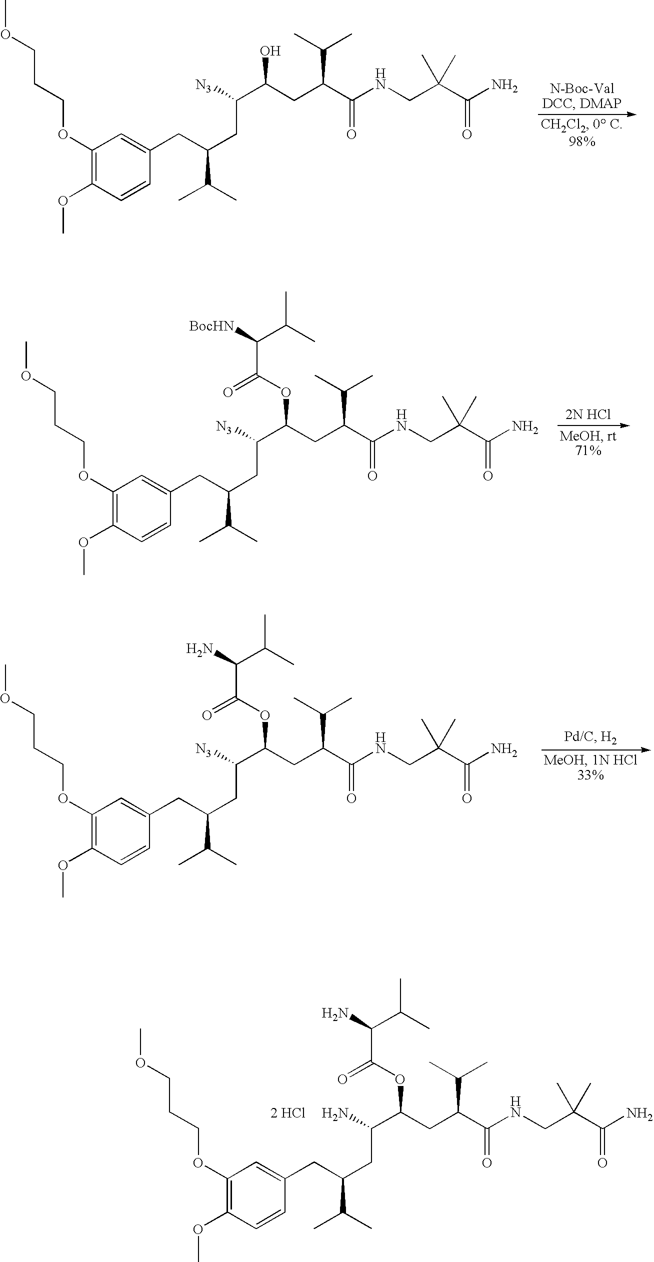

### EXAMPLE 1

### Acetic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester

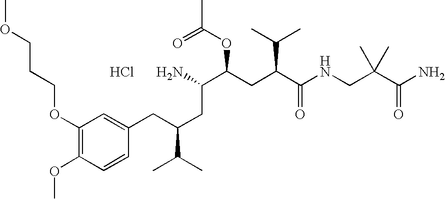

10% Pd/C (20 mg, Engelhard 4505) is added to a solution of acetic acid (1S,2S,4S)-2-azido-1-[(S)-2-(2-carbamoyl-2-methyl-propylcarbamoyl)-3-methyl-butyl]-4-[4-methoxy-3-(3-methoxy-propoxy)-benzyl]-5-methyl-hexl ester (55 mg, 0.09 mmol) in MeOH (3 ml) and 1 N HCl (1 ml) under Ar. The suspension then is stirred under a H₂ atmosphere for 5 h. The reaction mixture is filtered over Celite and the solvent is evaporated. Upon addition of diethyl ether the desired hydrochloride salt of acetic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester is obtained as a colorless solid.

MS (LC/MS): 594.3 [M+H]⁺

R_{f} (CH₂Cl₂/MeOH 9:1): 0.09.

The starting material can, for example, be prepared as follows:

### Acetic acid (1S,2S,4S)-2-azido-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester

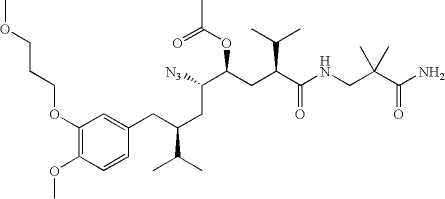

Triethyl amine (37 μl, 0.26 mmol, 1.5 eq), acetic anhydride (25 μl, 0.26 mmol, 1.5 eq) and DMAP (0.3 mg, 0.002 mmol, 0.01 eq) are added to a solution of (2S,4S,5S,7S)-5-azido-4-hydroxy-2-isopropyl-7-[4-methoxy-3-(3-methoxy-propoxy)benzyl]-8-methyl-nonanoic acid (2-carbamoyl-2-methyl-propyl)-amide (100 mg, 0.17 mmol) in THF (2 ml) under Ar. After stirring at rt for 3 h water is added and the mixture is extracted with ethyl acetate. Drying of the combined extracts (Na₂SO₄) and evaporation of the solvent affords the crude product which is purified by flash column chromatography (10 g SiO₂, CH₂Cl₂/MeOH 95:5) to yield the desired product as a colorless oil.

MS (LC/MS): 620.1 [M+H]⁺-R_{f}(CH₂Cl₂/MeOH 9:1): 0.36

The following compounds are prepared according to the experimental procedures described above:

### Propionic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester

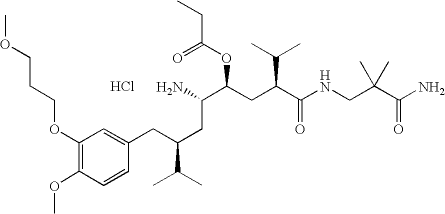

MS (LC/MS): 608.1 [M+H]⁺R_{f}(CH₂Cl₂₁MeOH 9:1): 0.20

### Propionic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester

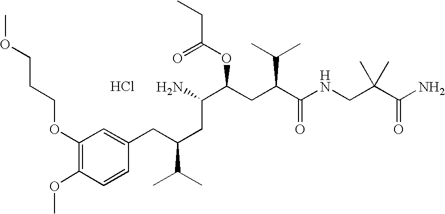

MS (LC/MS): 622.2 [M+H]⁺R_{f}(CH₂Cl₂/MeOH 9:1): 0.19

### Butyric acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester

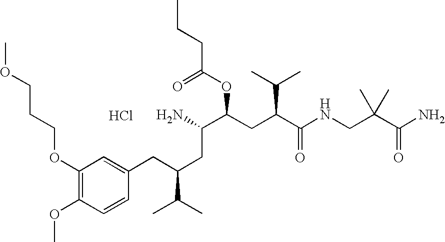

MS (LC/MS): 636.3 [M+H]⁺R_{f}(CH₂Cl₂/MeOH 9:1): 0.19

### Isobutyric acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester

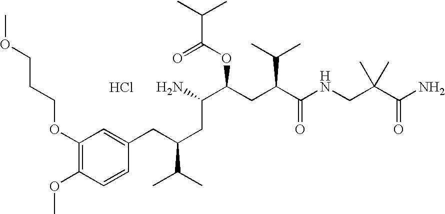

MS (LC/MS): 622.2 [M+H]⁺R_{f}(CH₂Cl₂/MeOH 9:1): 0.21

### 2,2-Dimethyl-propionic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester

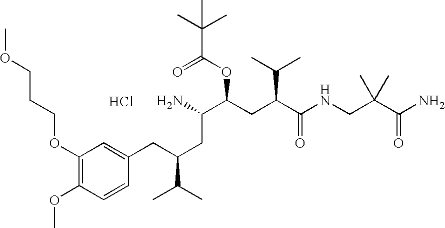

MS (LC/MS): 636.3 [M+H]⁺R_{f}(CH₂Cl₂/MeOH 9:1): 0.28

### EXAMPLE 2

### (S)-2-Amino-3-methyl-butyric acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester

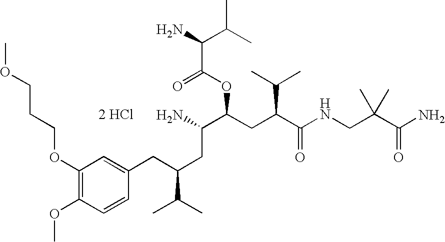

10% Pd/C (9 mg, Engelhard 4505) is added to a solution of (S)-2-Amino-3-methyl-butyric acid (1S,2S,4S)-2-azido-1-[(S-2)-(2-carbamoyl-2-methyl-propylcarbamoyl)-3-methyl-butyl]-4-[4-methoxy-3-(3-methoxy-propoxy)-benzyl]-5-methyl-hexyl ester (32 mg, 0.05 mmol) in MeOH (2 ml) and 1 N HCl (1 ml) under Ar. The suspension then is stirred under a H₂ atmosphere for 14 h. The reaction mixture is filtered over Celite and the solvent is evaporated. Upon addition of diethyl ether the product is isolated as a colorless solid.

MS (LC/MS): 651.3 [M+H]⁺-t_{R} (HPLC, C₁₈ column, 20-100% CH₃CN/H₂O): 3.27 min

The starting material can, for example, be prepared as follows:

### (S)-2-tert-Butoxycarbonylamino-3-methyl-butyric acid (1S,2S,4S)-2-azido-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester

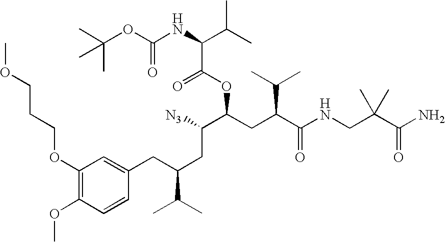

Dicyclohexylcarbodiimide (39 mg, 0.19 mmol, 1.1 eq) is added to a mixture of N-Boc valine (38 mg, 0.17 mmol), (2S,4S,5S,7S)-5-azido-4-hydroxy-2-isopropyl-7-[4-methoxy-3-(3-methoxy-propoxy)-benzyl]-8-methyl-nonanoic acid (2-carbamoyl-2-methyl-propyl)-amide (100 mg, 0.17 mmol) and 4-dimethylamino pyridine (21 mg, 0.17 mmol) in CH₂Cl₂ (2 ml) at 0° C. under Ar. The reaction mixture is stirred during 14 h, a saturated solution of NaHCO₃ is added and the mixture is extracted with CH₂Cl₂. The combined extracts are dried (Na₂SO₄) and the solvent is evaporated. Purification of the residue by flash column chromatography (10 g SiO₂, CH₂Cl₂ to CH₂Cl₂/MeOH 95:5) affords the desired product as a colorless oil. t_{R} (HPLC, C₁₈ column, 20-100% CH₃CN/H₂O): 6.70 min R_{f}(CH₂Cl₂/MeOH 9:1): 0.55

### (S)-2-Amino-3-methyl-butyric acid (1S,2S,4S)-2-azido-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester

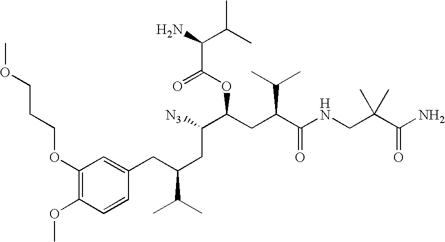

At 0° C. 2M HCl (0.47 ml, 0.93 mmol, 12 eq) is added to a solution of (S)-2-tert-Butoxycarbonylamino-3-methyl-butyric acid (1S,2S,4S)-2-azido-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester (60 mg, 0.077 mmol) in MeOH (2 ml). After stirring at RT for 14 h a saturated solution of NaHCO₃ is added and the mixtures is extracted with CH₂Cl₂. Drying (Na₂SO₄) and evaporation of the solvent affords the crude product which was subjected to flash column chromatography (10 g SiO₂, CH₂Cl₂/MeOH 9:1) to yield the product as a colorless oil.

MS (LC/MS): 677.4 [M+H]⁺-t_{R} (HPLC, C₁₈ column, 20-100% CH₃CN/H₂O): 4.67 min

The following compounds are prepared according to the experimental procedures described above:

### (S)-2-Amino-4-methyl-pentanoic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester

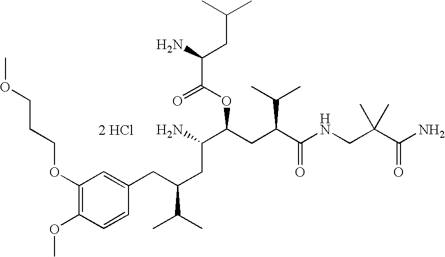

MS (LC/MS): 665.3 [M+H]⁺-t_{R} (HPLC, C₁₈ column, 20-100% CH₃CN/H₂O): 3.47 min

### (S)-2-Amino-3-phenyl-propionic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester

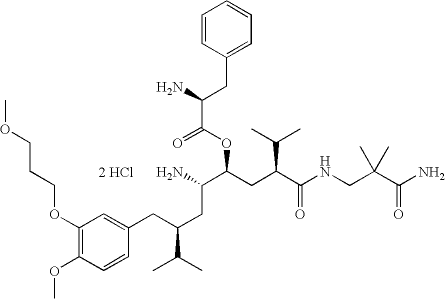

MS (LC/MS): 699.3 [M+H]⁺-t_{R} (HPLC, C₁₈ column, 20-100% CH₃CN/H₂O): 3.51 min

1. A compound of formula (I) 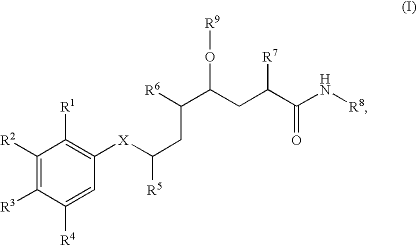
or a pharmaceutically acceptable salt thereof; wherein
R¹, R², R³, R⁴, independently of one another, are hydrogen; halogen; hydroxyl, C₁-C₇-alkanoyloxy, C₁-C₇-alkyl; or is
C₁-C₇-alkyl that is substituted by: halogen, cyano, hydroxy, C₁-C₇-alkanoyl-oxy, C₁-C₇-alkoxy, C₁-C₇-alkoxy that is substituted by halogen or by hydroxyl, C₂-C₇-alkenyloxy, C₃-C₇-cycloalkoxy, C₁-C₇-alkylthio, S-oxidized C₁-C₇-alkylthio, amino, N-mono-C₁-C₇-alkylamino, N,N-di-C₁-C₇-alkyl-amino, N—C₁-C₇-alkanoyl-amino, N—C₁-C₇-alkanesulfonyl-amino, amino that is N,N-disubstituted by C₂-C₇-alkylene, by unsubstituted or N′-C₁-C₇-alkyl- or N′-C₁-C₇-alkanoyl-aza-C₂-C₇-alkylene, by oxa-C₁-C₇-alkylene, by thia-C₁-C₇-alkylene or by S-oxidized thia-C₁-C₇-alkylene, free or esterified or amidated carboxy, C₃-C₇-cycloalkyl, aryl, heteroaryl, hydrogenated heteroaryl or by oxo; or is
C₁-C₇-alkoxy-C₂-C₇-alkenyl; or C₁-C₇-alkoxy; or is
C₁-C₇-alkoxy that is substituted by: halogen, cyano, hydroxyl, C₁-C₇-alkanoyl-oxy, C₁-C₇-alkoxy, C₁-C₇-alkoxy that is substituted by halogen or by hydroxy, C₂-C₇-alkenyloxy, C₃-C₇-cycloalkoxy, C₁-C₇-alkylthio, S-oxidized C₁-C₇-alkylthio, amino, N-mono-C₁-C₇-alkylamino, N,N-di-C₁-C₇-alkyl-amino, N—C₁-C₇-alkanoyl-amino, N—C₁-C₇-alkanesulfonyl-amino, amino that is N,N-disubstituted by C₂-C₇-alkylene, by unsubstituted or N′-C₁-C₇-alkyl- or N′-C₁-C₇-alkanoyl-aza-C₂-C₇-alkylene, by oxa-C₁-C₇-alkylene, by thia-C₁-C₇-alkylene or by S-oxidized thia-C₁-C₇-alkylene, free or esterified or amidated carboxy, C₃-C₇-cycloalkyl, aryl, heteroaryl, or by hydrogenated heteroaryl; or is
C₂-C₇-alkenyloxy; C₁-C₇-alkoxy-C₂-C₇-alkenyloxy; C₃-C₇-cycloalkoxy; C₁-C₇-alkanoyl; C₃-C₇-cycloalkyl; aryl; heteroaryl; or hydrogenated heteroaryl; or
R³ together with R₄ form C₂-C₇-alkylenedioxy or a fused-on benzo or cyclohexeno ring;
X is methylene; hydroxymethylene; O; NH; S; SO; or SO₂;
R⁵ is C₁-C₇-alkyl; C₂-C₇-alkenyl; C₃-C₇-Cycloalkyl; C₃-C₇-cycloalkyl-C₁-C₇-alkyl; aryl-C₁-C₇alkyl; heteroaryl-C₁-C₇-alkyl; aryl or heteroaryl;
R⁶ is amino; N-mono-C₁-C₇-amino; N,N-di-C₁-C₇-amino; N—C₁-C₇-alkanoyl-amino; N—C₁-C₇-alkanesulfonyl or represents a group of the formula —NR¹⁰COCHR¹¹NR¹²R¹³, the latter may be present either in the (D)-, (L)- or racemic (D, L)-configuration, but preferably in the L-form;
R⁷ is C₁-C₇-alkyl, C₂-C₇-alkenyl; C₃-C₇-cycloalkyl; C₃-C₇-cycloalkyl-C₁-C₇-alkyl; aryl-C₁-C₇-alkyl; heteroaryl-C₁-C₇-alkyl; aryl or heteroaryl;
R⁸ is hydrogen; C₁-C₇-alkyl; or is
C₁-C₇-alkyl that is substituted by: halogen, cyano, hydroxy, C₁-C₇-alkanoyl-oxy, C₁-C₇-alkoxy, C₁-C₇-alkoxy that is substituted by halogen or by hydroxyl, C₂-C₇-alkenyloxy, C₃-C₇-cycloalkoxy, C₁-C₇-alkylthio, S-oxidized C₁-C₇-alkylthio, amino, N-mono-C₁-C₇-alkylamino, N,N-di-C₁-C₇-alkyl-amino, N—C₁-C₇-alkanoyl-amino, N—C₁-C₇-alkanesulfonyl-amino, amino that is N,N-disubstituted by C₂-C₇-alkylene, by unsubstituted or N′-C₁-C₇-alkyl- or N′-C₁-C₇-alkanoyl-aza-C₂-C₇-alkylene, by oxa-C₁-C₇-alkylene, by thia-C₁-C₇-alkylene or by S-oxidized thia-C₁-C₇-alkylene, free or esterified or amidated carboxy, or is
C₁-C₇-alkanoyl; C₃-C₇-cycloalkyl, aryl, heteroaryl, hydrogenated heteroaryl; C₃-C₇-cycloalkyl;
aryl; heteroaryl or hydrogenated heteroaryl;
R⁹ represents C₁-C₇-alkanoyl, C₁-C₇-alkanesulfonyl or a group of the formula —COCHR¹⁴NR¹¹R¹² which may be present either in the (DY, (L)- or racemic (D, L)-configuration, but preferably in the L-form; or a group of the formula —CH₂O—COR¹⁵;
R¹⁰ is hydrogen; C₁-C₇-alkyl; C₃-C₇-cycloalkyl; C₃-C₇-cycloalkyl-C₁-C₇-alkyl; aryl-C₁-C₇-alkyl; heteroaryl-C₁-C₇-alkyl; aryl or heteroaryl;
R¹¹ is hydrogen; C₁-C₇-alkyl; aryl-C₁-C₇-alkyl; heteroaryl-C₁-C₇-alkyl; aryl or heteroaryl;
R¹² and R¹³, independently of another, are hydrogen; C₁-C₇-alkyl;
C₁-C₇-alkyl that is substituted by: halogen, C₃-C₇-cycloalkyl, aryl, heteroaryl, C₁-C₇-alkoxycarbonyl, C₁-C₇-alkylthio, by S-oxidized C₁-C₇-alkylthio, by aminocarbonyl, by N—C₁-C₇-alkanoyl-aminocarbonyl, by N—C₁-C₇-alkyl-aminocarbonyl; by N,N-di-C₁-C₇-alkyl-aminocarbonyl, or by aminocarbonyl that is disubstituted by C₂-C₇-alkylene; or are
C₃-C₇-cycloalkyl; aryl or heteroaryl;
R¹⁴ is hydrogen; C₁-C₇-alkyl; aryl-C₁-C₇-alkyl; heteroaryl-C₁-C₇-alkyl; aryl or heteroaryl; and
R¹⁵ is C₁-C₇-alkyl, aryl-C₁-C₇-alkyl; heteroaryl-C₁-C₇-alkyl; aryl or heteroaryl.

2. A compound according to claim 1 of formula (I) or a pharmaceutically acceptable salt thereof; wherein
R¹ is hydrogen, C₁-C₇-alkyl or C₁-C₇-alkoxy; R² is C₁-C₇-alkoxy or C₁-C₇-alkoxy-C₁-C₇-alkoxy; R³ is C₁-C₇-alkoxy or C₁-C₇-alkoxy-C₁-C₇-alkoxy; R⁴ is hydrogen, C₁-C₇-alkyl or C₁-C₇-alkoxy; R⁵ is C₁-C₇-alkyl; R⁶ is amino; R⁷ is C₁-C₇-alkyl; R⁹ is amino-carbonyl-C₁-C₇-alkyl; R⁹ is C₁-C₇-alkanoyl, a group of the formula —COCHR¹⁴NR¹¹R¹² which may be present either in the (D)-, (L)- or racemic (D, L)-configuration, but preferably in the L-form; or a group of the formula —CH₂O—COR¹⁵; and R⁴ is hydrogen, C₁-C₇alkyl or phenyl-C₁-C₄-alkyl; R¹² and R³, independently of one another, are hydrogen, C₁-C₇-alkyl or phenyl-C₁-C₄-alkyl; and R¹⁵ is C₁-C₇alkyl or phenyl-C₁-C₄-alkyl; and X is methylene.

3. A compound according to claim 1 of formula (I A) 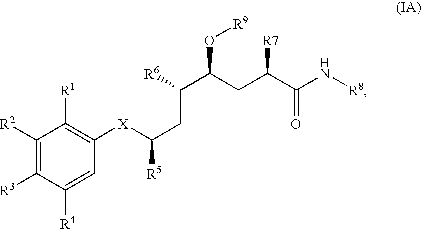
wherein the variables R¹ to R¹⁵ and X have all meanings as defined in claim 1; or a pharmaceutically acceptable salt thereof.

4. A compound according to claim 1 of formula (I A) or a pharmaceutically acceptable salt thereof, wherein
R¹ and R⁴ are hydrogen; R² is C₁-C₄-alkoxyl-C₁-C₄-alkoxy, such as 3-methoxy-propyloxy; R³ is C₁-C₄-alkoxy, such as methoxy; R⁵ and R₇, independently of one another, are C₁-C₇-alkyl, such as isopropyl; R⁶ is amino; R⁸ is aminocarbonyl-C₁-C₄-alkyl, such as 2-amino-2,2-dimethylethyl;
R⁹ is C₁-C₄-alkanoyl or a group of the formula —COCHR¹⁴NR¹²R¹³ wherein R¹⁴ is C₁-C₄-alkyl, such as isopropyl or isobutyl, or phenyl-C₁-C₂-alkyl, such as benzyl, R¹² and R¹³ are hydrogen and X is methylene.

5. A compound according to claim 4 of formula (I B) or a pharmaceutically acceptable salt thereof, wherein 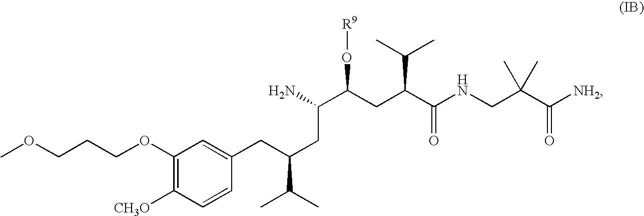
or a pharmaceutically acceptable salt thereof, wherein R⁹ is C₁-C₄-alkanoyl or a group of the formula —COCHR¹⁴NH₂ wherein R¹⁴ is C₁-C₄-alkyl, such as isopropyl or isobutyl, or phenyl-C₁-C₂-alkyl, such as benzyl.

6. A compound according to claim 1 or a pharmaceutically acceptable salt thereof selected from the group consisting of
acetic acid (1S,2S,4S)-2-amino-1-[(S)-2-(2-carbamoyl-2-methyl-propylcarbamoyl)-3-methyl-butyl]4-[4-methoxy-3-(3-methoxy-propoxy)benzyl]-5-methyl-hexyl ester;
propionic acid (1S,2S,4S)-2-amino-1-[(S)-2-(2-carbamoyl-2-methyl-propylcarbamoyl)-3-methyl-butyl]4-[4-methoxy-3-(3-methoxy-propoxy)-benzyl]-5-methyl-hexyl ester;
butyric acid (1S,2S,452-amino-1-[(S)-2-(2-carbamoyl-2-methyl-propylcarbamoyl)-3-methyl-butyl]4-[4-methoxy-3-(3-methoxy-propoxy)-benzyl]-5-methyl-hexyl ester;
isobutyric acid (1S,2S,4S)-2-amino-1-[(S)-2-(2-carbamoyl-2-methyl-propylcarbamoyl)-3-methyl-butyl]4-[4-methoxy-3-(3-methoxy-propoxy)-benzyl]-5-methyl-hexyl ester; M
2,2-dimethyl-propionic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester;
(S)-2-amino-3-methyl-butyric acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl Ester;
(S)-2-amino-4-methyl-pentanoic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester; and
(S)-2-amino-3-phenyl-propionic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester.

7. (canceled)

8. for the A method for the treatment of or prevention of or delay of progression to overt hypertension, congestive heart failure, cardiac hypertrophy, cardiac fibrosis, cardiomyopathy, postinfarction, (acute and chronic) renal failure, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, anxiety states and cognitive disorders comprising administering a therapeutically effective amount of the compound of claim 1.

9. A pharmaceutical composition comprising a compound according to claim 1 and a pharmaceutically acceptable carrier

10. A composition according to claim 9 further comprising at least one therapeutic agent selected from the group consisting of
(i) an AT₁-receptor antagonist or a pharmaceutically acceptable salt thereof,
(ii) an angiotensin converting enzyme (ACE) inhibitor or a pharmaceutically acceptable salt thereof,
(iii) a beta blocker or a pharmaceutically acceptable salt thereof,
(iv) a calcium channel blocker or a pharmaceutically acceptable salt thereof,
(v) an aldosterone synthase inhibitor or a pharmaceutically acceptable salt thereof,
(vi) an aldosterone receptor antagonist or a pharmaceutically acceptable salt thereof,
(vii) a dual angiotensin converting enzyme/neutral endopetidase (ACE/NEP) inhibitor or a pharmaceutically acceptable salt thereof,
(viii) an endothelin receptor antagonist or a pharmaceutically acceptable salt thereof,
(ix) a diuretic or a pharmaceutically acceptable salt thereof;
(x) a neutral endopeptidase (NEP) inhibitor or a pharmaceutically acceptable salt thereof;
(xi) an inhibitors of the Na—K-ATPase membrane pump or a pharmaceutically acceptable salt thereof;
(xii) an antidiabetic agent or a pharmaceutically acceptable salt thereof;
(xiii) a hypolipidemic agent or a pharmaceutically acceptable salt thereof; and
(xiv) an anti-obesity agent or a pharmaceutically acceptable salt thereof.

11. A compound according to claim 2 of formula (I A) 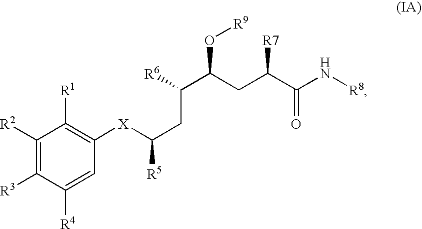
wherein the variables R¹ to R¹⁵ and X have all meanings as defined in claim 2; or a pharmaceutically acceptable salt thereof.

12. A compound according to claim 2 of formula (I A) or a pharmaceutically acceptable salt thereof, wherein
R¹ and R⁴ are hydrogen; R² is C₁-C₄-alkoxyl-C₁-C₄-alkoxy, such as 3-methoxy-propyloxy; R³ is C₁-C₄-alkoxy, such as methoxy; R⁵ and R⁷, independently of one another, are C₁-C₇-alkyl, such as isopropyl; R⁶ is amino; R⁸ is aminocarbonyl-C₁-C₄-alkyl, such as 2-amino-2,2-dimethylethyl;
R⁹ is C₁-C₄-alkanoyl or a group of the formula —COCHR¹⁴NR¹²R¹³ wherein R¹⁴ is C₁-C₄-alkyl, such as isopropyl or isobutyl, or phenyl-C₁-C₂-alkyl, such as benzyl, R¹² and R¹³ are hydrogen and X is methylene.

13. A compound according to claim 3 of formula (I A) or a pharmaceutically acceptable salt thereof, wherein
R¹ and R⁴ are hydrogen; R² is C₁-C₄-alkoxyl-C₁-C₄-alkoxy, such as 3-methoxy-propyloxy; R³ is C₁-C₄-alkoxy, such as methoxy; R⁵ and R⁷, independently of one another, are C₁-C₇-alkyl, such as isopropyl; R⁶ is amino; R⁸ is aminocarbonyl-C₁-C₄-alkyl, such as 2-amino-2,2-dimethylethyl;
R⁹ is C₁-C₄-alkanoyl or a group of the formula —COCHR¹⁴NR¹²R¹³ wherein R¹⁴ is C₁-C₄-alkyl, such as isopropyl or isobutyl, or phenyl-C₁-C₂-alkyl, such as benzyl, R¹² and R¹³ are hydrogen and X is methylene.

14. A compound according to claim 2 or a pharmaceutically acceptable salt thereof selected from the group consisting of
acetic acid (1S,2S,4S)-2-amino-1-[(S)-2-(2-carbamoyl-2-methyl-propylcarbamoyl)-3-methyl-butyl]4-[4-methoxy-3-(3-methoxy-propoxy)-benzyl]-5-methyl-hexyl ester;
propionic acid (1S,2S,4S)-2-amino-1-[(S)-2-(2-carbamoyl-2-methyl-propylcarbamoyl)-3-methyl-butyl]4-[4-methoxy-3-(3-methoxy-propoxy)-benzyl]-5-methyl-hexyl ester;
butyric acid (1S,2S,4S)-2-amino-1-[(S)-2-(2-carbamoyl-2-methyl-propylcarbamoyl-3-methyl-butyl]-4-[4-methoxy-3-(3-methoxy-propoxy)-benzyl]-5-methyl-hexyl ester;
isobutyric acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester; M
2,2-dimethyl-propionic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester;
(S)-2-amino-3-methyl-butyric acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl Ester;
(S)-2-amino-4-methyl-pentanoic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester; and
(S)-2-amino-3-phenyl-propionic acid (1S,2S,4S)-2-amino-1-[(S)-2-(2-carbamoyl-2-methyl-propylcarbamoyl)-3-methyl-butyl]-4-4-[4-methoxy-3-(3-methoxy-propoxy)-benzyl]-5-methyl-hexyl ester.

15. A compound according to claim 3 or a pharmaceutically acceptable salt thereof selected from the group consisting of
acetic acid (1S,2S,4S)-2-amino-1-[(S)-2-(2-carbamoyl-2-methyl-propylcarbamoyl)-3-methyl-butyl]4-[4-methoxy-3-(3-methoxy-propoxy)-benzyl]-5-methyl-hexyl ester;
propionic acid (1S,2S,4S)-2-amino-1-[(S)-2-(2-carbamoyl-2-methyl-propylcarbamoyl)-3-methyl-butyl]4-[4-methoxy-3-(3-methoxy-propoxy)-benzyl]-5-methyl-hexyl ester;
butyric acid (1S,2S,4S)-2-amino-1-[(S)-2-(2-carbamoyl-2-methyl-propylcarbamoyl)-3-methyl-butyl]4-[4-methoxy-3-(3-methoxy-propoxy)-benzyl]-5-methyl-hexyl ester;
isobutyric acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester; M
2,2-dimethyl-propionic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester;
(S)-2-amino-3-methyl-butyric acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl Ester;
(S)-2-amino-4-methyl-pentanoic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester; and
(S)-2-amino-3-phenyl-propionic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester.

16. A compound according to claim 4 or a pharmaceutically acceptable salt thereof selected from the group consisting of
acetic acid (1S,2S,4S)-2-amino-1-[(S)-2-(2-carbamoyl-2-methyl-propylcarbamoyl)-3-methyl-butyl]4-[4-methoxy-3-(3-methoxy-propoxy)-benzyl]-5-methyl-hexyl ester;
propionic acid (1S,2S,4S)-2-amino-1-[(S)-2-(2-carbamoyl-2-methyl-propylcarbamoyl)-3-methyl-butyl]4-[4-methoxy-3-(3-methoxy-propoxy)-benzyl]-5-methyl-hexyl ester;
butyric acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester;
isobutyric acid (1S,2S,4S)-2-amino-1-[(S)-2-(2-carbamoyl-2-methyl-propylcarbamoyl)-3-methyl-butyl]4-[4-methoxy-3-(3-methoxy-propoxy)-benzyl]-5-methyl-hexyl ester; M
2,2-dimethyl-propionic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester;
(S)-2-amino-3-methyl-butyric acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl Ester;
(S)-2-amino-4-methyl-pentanoic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester; and
(S)-2-amino-3-phenyl-propionic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester.

17. A compound according to claim 5 or a pharmaceutically acceptable salt thereof selected from the group consisting of
acetic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester;
propionic acid (1S,2S,4S)-2-amino-1-[(S)-2-(2-carbamoyl-2-methyl-propylcarbamoyl)-3-methyl-butyl]4-[4-methoxy-3-(3-methoxy-propoxy)-benzyl]-5-methyl-hexyl ester;
butyric acid (1S,2S,452-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester;
isobutyric acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester; M
2,2-dimethyl-propionic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester;
(S)-2-amino-3-methyl-butyric acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl Ester;
(S)-2-amino-4-methyl-pentanoic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester; and
(S)-2-amino-3-phenyl-propionic acid (1S,2S,4S)-2-amino-1-[(S)-2-(2-carbamoyl-2-methyl-propylcarbamoyl)-3-methyl-butyl]-4-[4-methoxy-3-(3-methoxy-propoxy)-benzyl]-5-methyl-hexyl ester.
